(19) United States
(12) Patent Application Publication
(10) Pub. No.: US 2016/0250058 A1
(43) Pub. Date: Sep. 1, 2016

(54) ORTHOPEDIC KNEEPAD

(71) Applicant: INDACO S.R.L., Filiano (PZ) (IT)

(72) Inventors: Massimo Frangi, Suvereto (IT); Roberta Frangi, Buguggiate (IT)

(21) Appl. No.: 15/028,423

(22) PCT Filed: Oct. 9, 2013
(86) PCT No.: PCT/IT2013/000276

§ 371 Date: Apr. 9, 2016

(51) Int. Cl. A61F 5/01

(52) U.S. Cl. CPC A61F 5/0106

(57) ABSTRACT

An orthopedic knee brace comprising first and second pockets made of a non-stretchable material which accommodate respective jointed support rods, a first portion of flexible and stretchable material that extends between the first and the second pockets, a second portion of flexible and stretchable material that extends from the second pocket to a free end, a first attachment member and a second attachment member made of non-stretchable material, which are located at said free end, the first pocket having first and second attachment members which are adapted to be respectively engaged by the first and second attachment members. The knee brace further comprises stabilization members for restricting the extension of the first and/or second portions of flexible and stretchable material.

The present invention relates to an orthopedic knee brace, i.e. a knee brace for use for retention of the knee joint.

Orthopedic knee braces have the purpose of restricting the movement of the knee joint and are often used in post-surgery for protecting the joint from sprain injuries, inappropriate rotation, voluntary or involuntary excessive flexion or extension, by transferring the load on jointed metal bars.

Orthopedic knee braces are classified into two main types: “closed” knee braces, that have to be fitted on the leg from the foot, and “open” knee braces, that may be worn directly level with the knee and then closed therearound.

Usually, “closed” knee braces provide improved comfort to the user, as they uniformly encircle the joint. On the other hand, this type of knee joints is particularly difficult to wear. A tubular sock with jointed metal bars is not easy to wear on an injured limb under treatment.

“Open” knee braces are more easy to wear but provide low user comfort levels. These knee braces usually comprise lateral portions with the above mentioned jointed bars and elastic or non-elastic belts that are designed to be fastened above the knee (on the thigh) or below the knee (on the tibia) to hold the jointed metal bars in position. These belts tend to cause injury to leg muscles, as they must be tightly fastened to prevent displacement of the jointed metal bars.

In addition, in both types of orthopedic knee braces, the metal bars must be firmly attached to the leg portions above and below the knee respectively. Otherwise, the knee might not carry out its movement restricting action on the joint as desired.

In prior art knee braces, of either “closed” or “open” type, the problem of firm attachment of the jointed metal bars to the leg has not been adequately solved, when maintaining a high user comfort.

In light of the above prior art, the object of the present invention is to provide an orthopedic knee brace that affords an accurately controlled movement of the user's knee while providing a high comfort level for the user.

Another object is to provide a knee joint that can be quickly and easily fastened to the user's leg in a proper position relative to the leg.

According to the present invention, the above objects are fulfilled by a knee joint as defined in one or more of the annexed claims.

Further features and advantages of the present invention will appear from the following detailed description of a preferred, non-limiting embodiment, which is illustrated without limitation in the annexed drawings, in which:

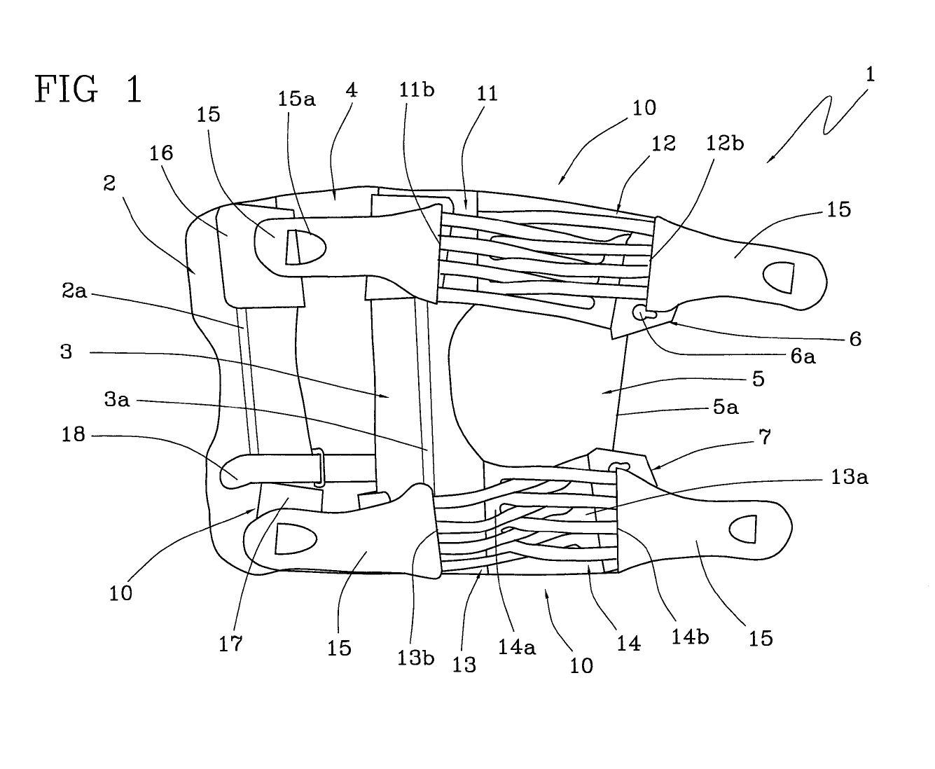

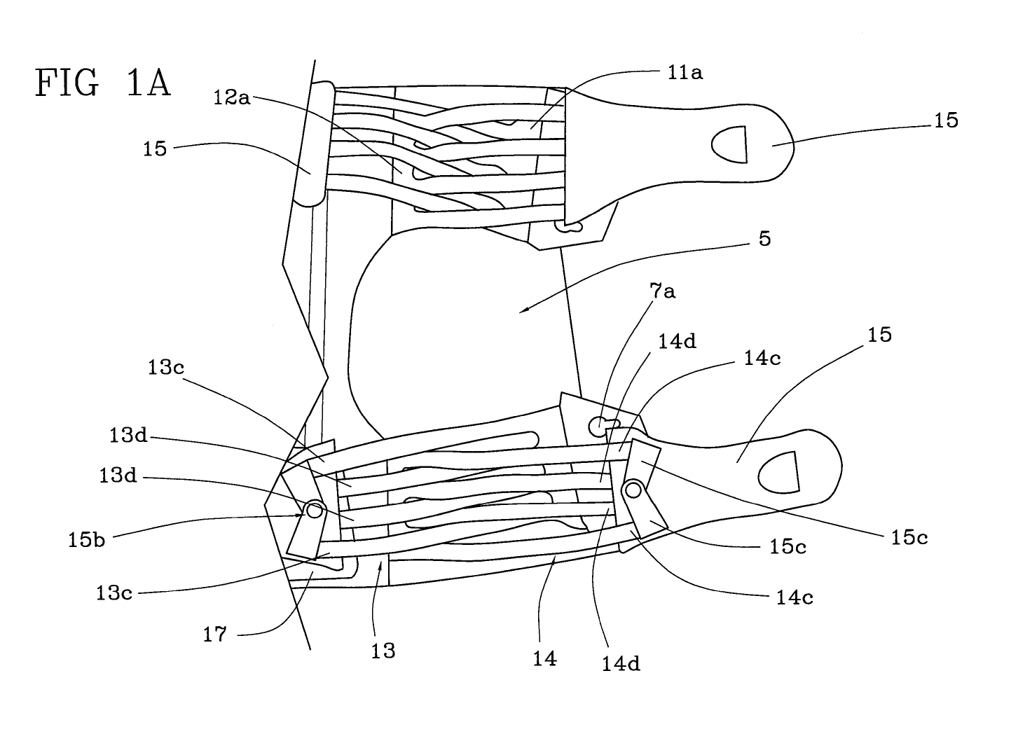

FIG. 1 is a plan view of a knee brace of the invention;

FIG. 1A is an enlarged view of a few details of the knee brace of FIG. 1;

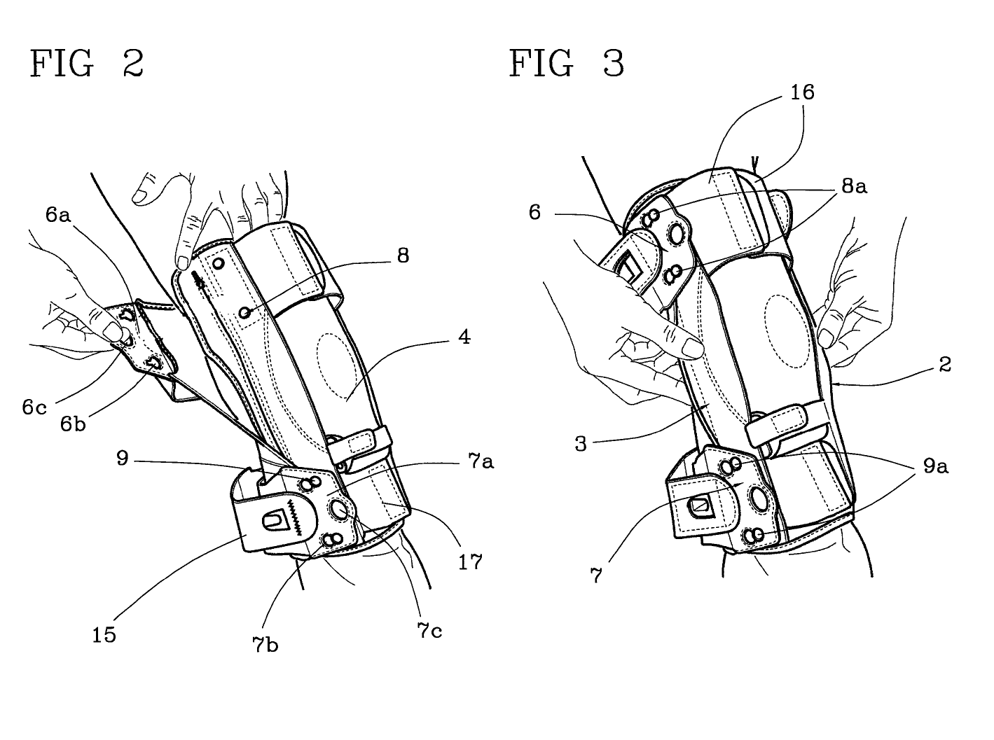

FIGS. 2 to 5 are perspective views of the knee brace of FIG. 1 as it is being worn on the leg of a user;

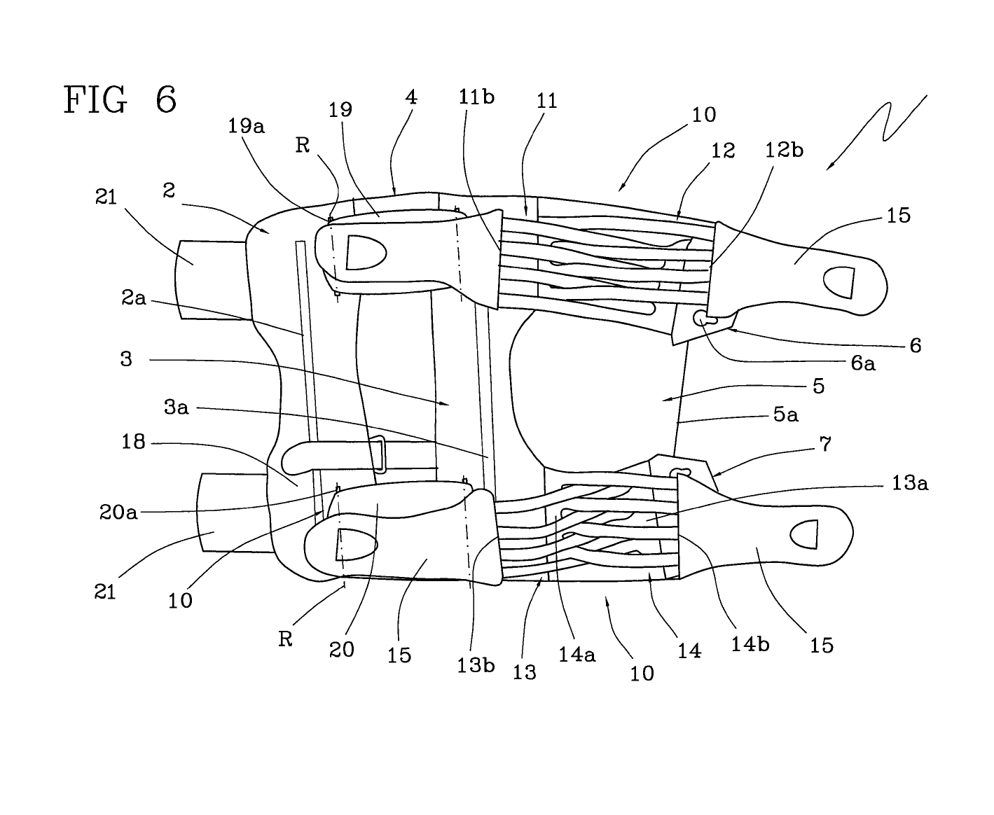

FIG. 6 is a plan view of a variant of the knee brace of FIG. 1; and

FIGS. 2 to 7 are perspective views of the knee brace of FIG. 6 as it is being worn on the leg of a user.

FIG. 1 shows an orthopedic knee brace 1 according to the present invention. Particularly, FIG. 1 shows the knee brace 1 in an open, unworn state.

The knee brace 1 comprises first 2 and second 3 pockets made of a non-stretchable material, which are designed to accommodate respective jointed support rods (not shown). As used herein, the term non-stretchable material is intended to designate an inelastic material which does not extend when it is pulled by a user during normal use of the knee brace. Likewise, the term stretchable material is intended to designate an elastic material which extends by a user-perceivable length as a user pulls it. The stretch material of the present invention is a material that can extend to twice its length during use.

Also, the term flexible material is intended to designate a stretchable or non-stretchable material that can be bent and twisted when handled by a user. An exemplary flexible material is a fabric. The term rigid manner is intended to designate a material that is not appreciably deformed during normal use of the knee brace.

The first 2 and second 3 pockets are made of a flexible material and are designed to contain the above mentioned jointed rods, which are made of a rigid material and have the purpose of restricting the movement of the knee joint. The two pockets have a main direction of extension that coincides with the main direction of extension of the jointed rods. As used herein, the term pocket shall be intended to designate any support for retaining jointed rods, whether such jointed rods are hidden in the pocket or exposed out of it. Advantageously, each pocket is closed by its respective hinge 2a, 3a, such that the jointed rods are entirely held within the pockets, and may be removed therefrom. Fastener members (not shown) are provided in the pockets to retain the ends of the jointed rods, thereby ensuring that they hold predetermined positions in their pockets.

A first portion 4 of flexible and stretchable material extends between the first 2 and second 3 pockets. A second portion 5 of flexible and stretchable material extends from the second pocket 3 to a free end 5a. The first 4 and second 5 portions of flexible and stretchable material are firmly attached to the pockets 2, 3, as mentioned above, to form a single body. Each portion of flexible and stretchable material is attached to its respective pocket along one edge, such that the single body defined by the assembly of pockets and portions of flexible and stretchable material, forms an alternation of a first pocket 2, a first portion 4 of flexible and stretchable material, a second pocket 3, a second portion 5 of flexible and stretchable material. It shall be noted that the knee brace is of the “open” type, i.e. has the form of a strip that has not been closed into a tube, in the unworn state.

Advantageously, the knee brace 1 comprises a first attachment member 6 and a second attachment member 7 made of non-stretchable material, which are located at the free end 5a of the second portion 5 of flexible and stretchable material. The first 6 and second 7 attachment members are made of a rigid material, and have the purpose of closing the knee brace 1 into a tube around the knee of the user (as shown in FIGS. 2 and 3). The first and second attachment members are designed to join together in respective predetermined positions on the first pocket 2, to define a tubular body that encircles the user's knee. For this purpose, the knee brace 1 comprises first 8 and second 9 coupling members (as shown in FIGS. 2 and 3) located on the first pocket 2. The coupling members 8, 9 are placed in preset and immovable positions on the first pocket 2 and are designed to be engaged by the attachment members 6, 7.

In the preferred embodiment of the invention, the attachment members 6, 7 comprise a plate 6a, 7a, with slots 6b, 7b formed therein. A pull member 6c, 7c is further provided on the plate 6a, 7a to allow a user to firmly grasp the plate. In the preferred embodiment of the invention, the pull member 6c, 7c consists of a hole that is designed to be engaged by a user's finger (see FIG. 2). The coupling members comprise respective mushroom buttons 8a, 9a, that are designed to be engaged by the above mentioned slots, to stably retain them.

The knee brace 1 further comprises stabilization members 10 for restricting the extension of the first 4 and/or second 5 portions of flexible and stretchable material.

As mentioned above, the attachment members 6, 7 and the coupling members 8, 9 have the purpose of closing the knee brace into a tube around the knee of the user. The portions 4, 5 of flexible and stretchable material allow the knee brace to adhere to the joint and to properly position the jointed rods at the sides of the knee (as described below in further detail). The stabilization members 10 allow the knee brace to be stabilized in the best operating position and prevent any further extension of the portions 4, 5 of flexible and stretchable material. It shall be noted that the stabilization members 10 are not designed to exert elastic compression forces on the leg of the user, i.e. are not designed to be elastically deformed during use of the knee brace. For this purpose, the stabilization members 10 are made of a non-stretchable material.

The stabilization members 10 are in such positions on the knee brace as to act on leg portions above and below the knee, when the knee brace is worn.

The stabilization members 10 comprise (see FIGS. 1 and 1A) a first plurality 11 of first straps, which are sequentially connected by a first end 11a to the first attachment members 6 and a first plurality 12 of second straps which are sequentially connected by a first end 12a to the second pocket 3. The first straps 11 alternate with the second straps 12 for mutual attachment. The first plurality of first 11 and second 12 straps defines a maximum extension of the second portion 5 of the flexible and stretchable material. The second ends 11b of the first straps 11 opposite to the first ends 11a are interconnected and adapted to be fixed to an outer surface of the first pocket 2 or the first attachment members 6. The second ends 12b of the second straps 12 opposite to the first ends 12a are interconnected and adapted to be fixed to an outer surface of the first pocket 2 or the first attachment members 6.

The stabilization members 10 comprise a second plurality 13 of first straps, which are sequentially connected by a first end 13a to the second attachment members 7 and a second plurality of second 12 straps which are sequentially connected by a first end 14a to the second pocket 3. The first straps 13 alternate with the second straps 14 as described above. The second plurality of first 13 and second 14 straps defines a maximum extension of the second portion 5 of the flexible and stretchable material. The second ends 13b of the first straps 13 opposite to the first ends 13a are interconnected and adapted to be fixed to an outer surface of the first pocket 2 or the second attachment members 7. The second ends 14b of the second straps 14 opposite to the first ends 14a are interconnected and adapted to be fixed to an outer surface of the first pocket 2 or the second attachment members 7.

The second ends 11b, 13b of the first straps of the first 11 and second 13 pluralities of first straps and the second ends 12b, 14b of the second straps of the first 12 and second 14 pluralities of second straps are adapted to be fixed in non-predetermined positions on the above mentioned outer surface, according to the anatomy of the user's leg.

The first plurality of first 11 and second 12 straps is operable over an area of the knee brace that is designed to be located above the knee of a user, and the second plurality of first 13 and second 14 straps is operable on an area of the knee brace that is designed to be located below the knee of a user.

As better shown below, the provision of such alternating straps affords easy stabilization of the knee brace on the leg, and ensures that the knee brace is in its proper position relative to the leg.

Preferably, at least three, preferably four straps are provided for each plurality of first and second straps. The straps preferably have a width of less than 2 cm. The straps have a length that is at least equal to the transverse dimension of the second portion 5 of stretchable and flexible material when measured in its unstretched state from the second pocket 3 to the free end 5a. The straps are oriented substantially transverse to the axial direction of extension of the jointed rods.

Pull members 15 made of non-stretchable material are connected to the second ends 11b, 13b of the first straps of the first 11 and second 13 pluralities of first straps and to the second ends 12b, 14b of the second straps of the first 12 and second 14 pluralities of second straps. Such pull members 15 comprise a Velcro portion for firm attachment thereof to the above mentioned outer surface. Furthermore, the pull members have respective holes 15a that are designed to be engaged by a finger of the user, for easy and ready grasp by a user when wearing the knee brace.

It shall be noted that the second ends 13b, 14b of the second plurality of first and second straps comprise end straps 13c, 14c and central straps 13d, 14d (see FIG. 1A). The central straps 13d, 14d are firmly attached to their respective pull members 15, whereas the end straps 13c, 14c are adapted to be fixed in a plurality of positions on their respective pull members 15. Particularly, the end straps 13c, 14c are integral with tabs 15b (only shown in FIG. 1A for illustration purposes), which are in turn movable relative to the pull member 15. The tabs 15b are also designed to be stably but removably attached to the pull member 15 in a plurality of positions. In the preferred embodiment of the invention, the tabs 15b are hinged to their respective pull members and have two projections 15c relative to the hinge point, at whose ends they are integral with the end straps 13c, 14c. Preferably, each of the two projections 15c of each tab 15b is jointed relative to the hinge point, such that each end strap is adapted to be positioned on its respective pull member, independently of the other end strap.

In a first embodiment of the invention, the stabilization members 10 also comprise a first pair of tabs 16 made of a flexible and non-stretchable material, that have a free edge and respective edges attached to the first pocket 2 and the second pocket 3. These tabs 16 are designed to be mutually attached at their respective free ends to define a maximum extension limit for the first portion 4 of flexible and stretchable material. The stabilization members also comprise a second pair of tabs 17 having a free edge and respective edges attached to the first pocket 2 and the second pocket 3. The tabs 17 of said second pair of tabs 117 are designed to be mutually attached at their respective free ends to define a maximum extension limit for the first portion 4 of flexible and stretchable material. The first pair of tabs 16 acts upon the thigh of the user, and the second pair of tabs 17 acts upon the tibia of the user, when the knee brace is worn.

The knee brace 1 further comprises a belt 18 operable between the first pocket 2 and the second pocket 3, which is placed immediately below the knee to assist in holding the knee brace in position during use.

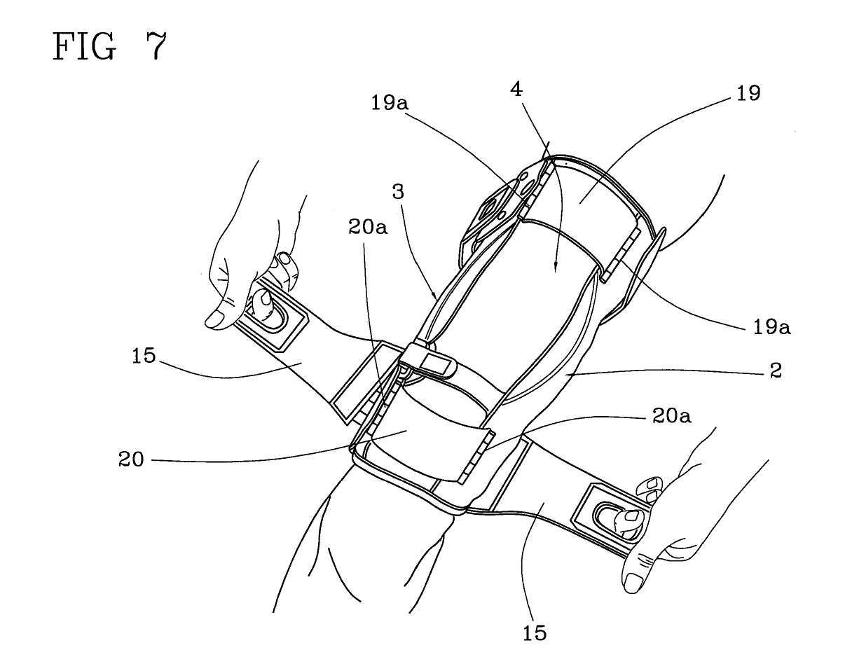

In a second embodiment of the invention (as shown in FIGS. 6 and 7), the stabilization members 10 comprise a first bracket 19 made of a rigid material, which extends between the first 2 and second 3 pockets, over the first portion 4 of flexible and stretchable material. This first bracket 19 has a curved shape, with its concavity facing the first portion of flexible and stretchable material.

In the second embodiment, the stabilization members 10 comprise a second bracket 20 made of a rigid material, which extends between the first 2 and second 3 pockets, over the first portion 4 of flexible and stretchable material. This second bracket has a curved shape, with its concavity facing the first portion of flexible and stretchable material. In the second embodiment of the invention, the above mentioned pairs of tabs 16, 17 are omitted, as their function is accomplished by the first and second brackets. Thus, the brackets 19, 20 have the purpose of restricting the extension of the first portion 4 of flexible and stretchable material. The first bracket 19 acts upon the thigh of the user, and the second bracket 20 acts upon the tibia of the user, when the knee brace is worn. The two brackets 19, 20 are a rigid and non-stretchable abutment for the limb portions upon which they act, whereby this knee brace is stiffer than the knee brace with the pairs of tabs 16, 17.

Preferably, the first bracket 19 is attached to the first and second pockets along respective hinges 19a having substantially parallel axes of rotation R. The axes of rotation R are also substantially parallel to the main extension of the jointed rods. Likewise, the second bracket 20 is attached to the first and second pockets along respective hinges 20a having substantially parallel axes of rotation R. The axes of rotation R are also substantially parallel to the main extension of the jointed rods. This connection between the brackets and the pockets afforde easier wearing of the knee brace. Each bracket 19, 20 has such transverse dimensions, i.e. substantially transverse to the direction of extension of the jointed rods, as to allow the pockets to be placed at the sides of the knee once the knee brace is worn. Advantageously, the respective concavities of the two brackets 19, 20 have different shapes. This will allow the concavity of each bracket to perfectly conform with the part of the limb upon which it acts. Particularly, the concavity of the second bracket 20 is shallower, i.e. has a lower radius of curvature, than the concavity of the first bracket 19, to reflect the larger circumference of the thigh as compared with the tibia.

Furthermore, in both cases of the brackets 19, 20 being hinged to the pockets 2, 3 and being rigidly attached to the pockets, the attachment between the brackets 19, 20 and the pockets 2, 3 is removable, to allow removal of the brackets 19,20 and replacement thereof with brackets of different sizes. By this arrangement, the knee joint may be adapted to limbs of different sizes without requiring the replacement of the entire knee brace. This possibility is particularly advantageous when considering that, during rehabilitation the limb of a user changes its size as it recovers its tonus.

The knee brace 1 further comprises at least one pair, preferably two pairs of shoulders 21, which are designed to be placed at the back of the thigh and tibia, when the knee brace is being worn. These shoulders have the purpose of encircling and supporting the back of the thigh and the tibia. Each shoulder 21 is attached to a respective pocket 2, 3 (FIG. 6 only shows the shoulders attached to the first pocket 2) and extends away from it. Particularly, a shoulder of the first pair of shoulders is integral with the first pocket 2 and the other shoulder of the first pair of shoulders is integral with the second pocket 3. Likewise, a shoulder of the second pair of shoulders is integral with the first pocket 2 and the other shoulder of the second pair of shoulders is integral with the second pocket 3.

The shoulders 21 have a first end attached to their respective pocket and a free end facing away from the first end. Each shoulder has a substantially curved profile with its concavity facing the limb of the user when the knee brace is being worn. The free ends of a pair of shoulders are in mutually facing and possibly partially overlapped relationship when the knee brace is being worn.

The shoulders attached to the second pocket 3 are placed below the first 12 and second 14 plurality of second straps. Particularly, the shoulders attached to the second pocket extend substantially at and below the first end 12a, 14a of the first 12 and second 14 plurality of second straps.

In one embodiment of the knee brace, not shown, the shoulders 21 are attached to their respective pockets along respective hinges whose axes of rotation are substantially parallel to each other and substantially parallel to the main extension of the jointed rods. In the embodiment as shown in the annexed figures, the shoulders are rigidly attached to the pockets and to portions of the jointed rods thereby structurally forming a single body with the respective jointed rods.

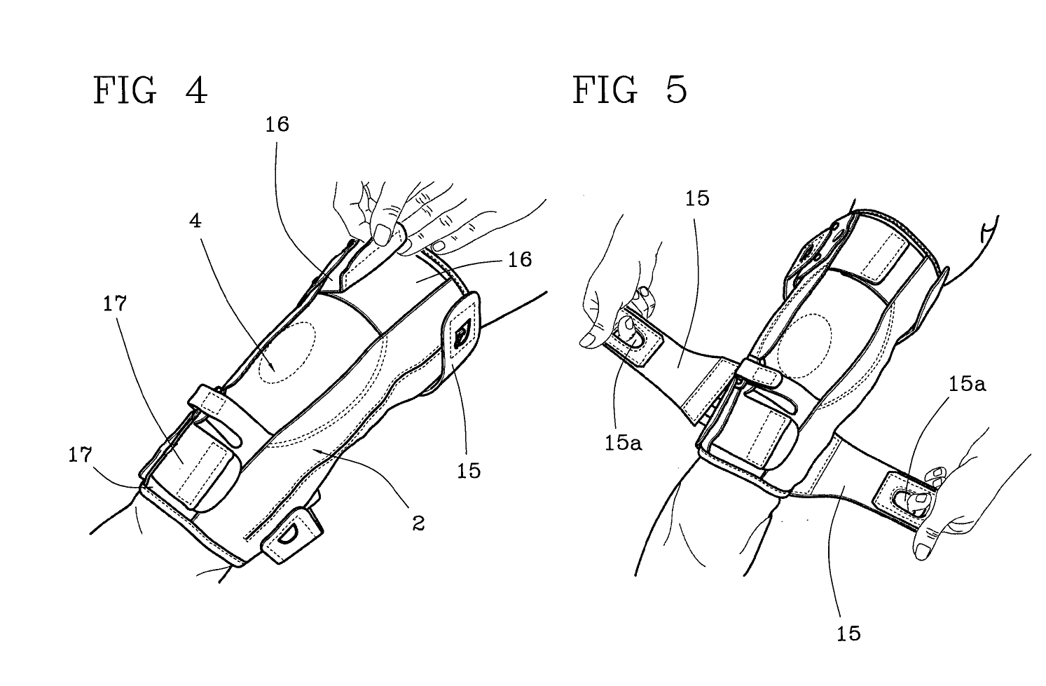

In operation, the knee brace is wrapped around the knee of a user, with the first portion 4 of flexible and stretchable material positioned at the front of the knee, the second portion 5 of flexible and stretchable material at the back of the knee and the two pockets 2, 3 at the sides of the knee (see FIG. 2). Then, the attachment members 6, 7 are engaged on the attachment members 8, 9 (see FIG. 2) such that the knee brace is closed into a tube around the knee. In this configuration, the knee brace 1 firmly abuts the knee and fine position adjustment may be carried out. Thus, when acting upon the pockets 2, 3, the portions 4, 5 of flexible and stretchable material follow the movements of the pockets 2, 3. By this arrangement, the jointed rods may be both placed into the proper position (see FIG. 3) by simultaneously acting upon both pockets. Once the knee brace has been properly positioned, the position is stabilized by actuating the stabilization members 10. Particularly, the first 16 and second 17 tabs are coupled together, such that the first portion 4 of flexible and stretchable material can no longer extend (see FIG. 4). Then, the second pluralities of first 13 and second 14 straps may be grasped, pulled and fastened at the front of the knee using the members 15 (see FIG. 5). In this step, the end straps 13c, 14c are placed on their respective pull member 15 to be more tightly fastened than the central strap 13d, 14d, for improved conformation to the anatomy of the wrapped muscle and improved fixation of the knee brace on the limb. Likewise, the first pluralities of first 11 and second 12 straps are grasped and pulled using the members 15. The second portion of flexible and stretchable material is locked in position and prevented from further extension.

Now, the knee brace is firmly fastened around the knee.

In the embodiment of the knee brace (as shown in FIGS. 6 and 7) in which the retention members 10 comprise the brackets 19, 20, after engagement of the attachment members 6, 7 on the coupling members 8, 9 (see FIG. 7), the knee brace 1 firmly abuts the knee and the brackets 19, 20 fix the position of the knee brace. The wearing process continues as described above.

Now, the purposes of the present invention appear to be fulfilled. Particularly, the knee brace as provided herein ensures high comfort. Furthermore, the knee brace is easy to fasten into a desired position.

The clamping force is evenly arranged along the longitudinal extent of the knee brace, due to the provision of the plurality of straps.

Furthermore, firm attachment of the jointed rods to the leg is ensured. This will provide reliable control of the maximum admitted bending angles for the patient's knee.

Those skilled in the art will obviously appreciate that a number of changes and variants may be made to the arrangements as described hereinbefore to meet incidental and specific needs, without departure from the scope of the invention, as defined in the following claims.

1: An orthopedic knee brace comprising first and second pockets made of a non-stretchable material which are designed to accommodate respective jointed support rods, a first portion of flexible and stretchable material that extends between the first and the second pockets, a second portion of flexible and stretchable material that extends from the second pocket to a free end, a first attachment member and a second attachment member made of non-stretchable material, which are located at said free end of the second portion of flexible and stretchable material, opposite to the end attached to the second pocket, said first pocket having first and second attachment members which are adapted to be respectively engaged by said first and second attachment members to hold the latter in predetermined positions relative to the first pocket; the orthopedic knee brace further comprising stabilization members for restricting the extension of the first and/or second portions of flexible and stretchable material.

2: An orthopedic knee brace as claimed in claim 1, wherein said stabilization members are made of a non-stretchable material.

3: A knee brace as claimed in claim 1, wherein said stabilization members comprise a first plurality of first straps sequentially connected at one first end to said first attachment members and a first plurality of second straps sequentially connected at one first end to the second pocket; the first straps alternating with the second straps; said first plurality of first and second straps defining a maximum limit to the extension of the second portion of flexible and extensible material; second ends of the first straps, which face away from the first ends, being adapted to be fixed to an outer surface of the first pocket or the second pocket or the first attachment members; second ends of the second straps, which face away from the first ends being adapted to be fixed to an outer surface of the first pocket or the second pocket or the first attachment members.

4: A knee brace as claimed in claim 1, wherein said stabilization members comprise a second plurality of first straps sequentially connected at one first end to said second attachment members and a second plurality of second straps sequentially connected at one first end to the second pocket; the first straps alternating with the second straps; said second plurality of first and second straps defining a maximum limit to the extension of the second portion of flexible and stretchable material; second ends of the first straps, which face away from the first ends, being adapted to be fixed to an outer surface of the first pocket or the second pocket or the second attachment members; second ends of the second straps, which face away from the first ends being adapted to be fixed to an outer surface of the first pocket or the second pocket or the second attachment members.

5: An orthopedic knee brace as claimed in claim 3, wherein the second ends of the first straps of the first and second pluralities of first straps and the second ends of the second straps of the first and second pluralities of second straps are adapted to be fixed in non-predetermined positions on said outer surface, according to the anatomy of the user's leg.

6: A knee brace as claimed in claim 4, wherein said first plurality of first and second straps is operable over an area of the knee brace that is designed to be located above the knee of a user, and said second plurality of first and second straps is operable on an area of the knee brace that is designed to be located below the knee of a user.

7: An orthopedic knee brace as claimed in claim 1, wherein said first and second attachment members comprise a plate made of a non-stretchable material, having slots designed for engagement with respective buttons of the first and second attachment members.

8: An orthopedic knee brace as claimed in claim 5, comprising pull members made of a non-stretchable material, which are connected to the second ends of the first straps of the first and second pluralities of first straps and to the second ends of the second straps of the first and second pluralities of second straps; said pull members comprising a Velcro portion for firm fixation to the outer surface of the first pocket or the second pocket or the first or second attachment members, or to themselves, according to the size of the user.

9: A knee brace as claimed in claim 3, wherein said plurality of first and second straps have a length that is at least equal to the transverse dimension of the second portion of stretchable and flexible material when measured in its unstretched state from the second pocket to the free end.

10: An orthopedic knee brace as claimed in claim 1, wherein, in operation, the first pocket and the second pocket are designed to be positioned at the sides of the knee of a user, the first portion of flexible and stretchable material is designed to be positioned at the front of the knee and the second portion of flexible and stretchable material is designed to be positioned at the back of the knee.

11: An orthopedic knee brace as claimed in claim 1, wherein said stabilization members comprise a first pair of tabs, having a free edge and respective edges attached to the first pocket and the second pocket; the tabs of said first pair of tabs being adapted to be mutually attached at their respective free ends to define a maximum extension limit for the first portion of flexible and stretchable material.

12: An orthopedic knee brace as claimed in claim 11, wherein said stabilization members comprise a second pair of tabs, having a free edge and respective edges attached to the first pocket and the second pocket; the tabs of said second pair of tabs being adapted to be mutually attached at their respective free ends to define a maximum extension limit for the first portion of flexible and stretchable material.

13: A knee brace as claimed in claim 1, wherein said stabilization members comprise a first bracket made of a rigid material, which extends between the first and second pockets, over the first portion of flexible and stretchable material.

14: A knee brace as claimed in claim 13, wherein said first bracket has a curved shape, with its concavity facing the first portion of flexible and stretchable material.

15: A knee brace as claimed in claim 13, wherein said stabilization members comprise a second bracket made of a rigid material, which extends between the first and second pockets, over the first portion of flexible and stretchable material, said first bracket being designed to abut the thigh of a user, and said second bracket being designed to abut the tibia of a user.

16: A knee brace as claimed in claim 15, wherein said second bracket has a curved shape, with its concavity facing the first portion of flexible and stretchable material.

17: An orthopedic knee brace as claimed in claim 15, wherein said first bracket and said second bracket are attached to the first and second pockets along respective hinges whose axes of rotation are substantially parallel to each other and substantially parallel to the main extension of the jointed rods.

18: An orthopedic knee brace as claimed in claim 13, comprising two pairs of shoulders, which are designed to be placed at the back of the thigh and tibia of a user respectively.

19: An orthopedic knee brace as claimed in claim 18, wherein each shoulder is attached to a respective pocket and extends away from it.

20: An orthopedic knee brace as claimed in claim 19, wherein each shoulder has a first end attached to its respective pocket and a free end facing away from the first end; each shoulder having a substantially curved profile with its concavity facing the limb of the user, when the knee brace is being worn.
